(19) United States
(12) Patent Application Publication
(10) Pub. No.: US 2010/0255546 A1
(43) Pub. Date: Oct. 7, 2010

(54) NUCLEIC ACID AMPLIFICATION METHOD

(76) Inventors: Chihiro Uematsu, Kawasaki (JP); Yukie Nakashima, Shiki (JP)

Correspondence Address: MATTINGLY & MALUR, P.C., 1800 DIAGONAL ROAD, SUITE 370, ALEXANDRIA, VA, 22314, US

(21) Appl. No.: 11/949,330

(22) Filed: Dec. 3, 2007

(30) Foreign Application Priority Data
Dec. 5, 2006 (JP) 2006-328211

(51) Int. Cl. C12P 19/34

(52) U.S. Cl. 435/91.2

(57) ABSTRACT

Disclosed is a novel isothermal nucleic acid amplification method enabling inexpensive and simple and easy detection. The method includes introducing nicking enzyme recognition sequences into an analysis target nucleic acid using nicking enzyme recognition sequence-containing primers and isothermally amplifying a specific region of the target nucleic acid using the primers, a nicking enzyme and a DNA polymerase having strand displacement activity.

### CLAIM OF PRIORITY

The present application claims priority from Japanese application JP 2006-328211 filed on Dec. 5, 2006, the content of which is hereby incorporated by reference into this application.

### FIELD OF THE INVENTION

The present invention relates to a nucleic acid (DNA or RNA) amplification method. More particularly, it relates to a novel isothermal nucleic acid amplification method which uses a primer(s) having a nicking enzyme recognition site and a DNA polymerase having strand displacement activity.

### BACKGROUND OF THE INVENTION

In studying vital phenomena, the technique of DNA and RNA amplification is used for various purposes. For example, the competitive PCR method (Non-patent document 1: A. Wang, et al., Proc. Natl. Acad. Sci. USA, 86, 9717-9721 (1989)) and the real-time PCR method (Non-patent document 2: K. Edwards, et al., Journal of Antimicrobial Chemotherapy, 54, 968 (2004)), among others, are known as methods for gene expression analysis and gene expression level quantification. These methods all employ the PCR (polymerase chain reaction) technique (Non-patent document 3: R. K. Saiki, et al., Science, 239, 487-491 (1988)), which is now a common nucleic acid amplification method, to determine the gene expression level based on the amount of the gene amplified.

The nucleic acid amplification methods for the above-mentioned analytical purposes comprise three steps, namely the steps of denaturation of a double-stranded DNA to single-stranded DNAs, annealing of primers to a single-stranded DNA, and complementary strand extension from the primers, or two steps, namely the steps of such denaturation and strand extension, and it is essential to repeat a cycle from a higher temperature step to a lower temperature step. For carrying out such steps, it is necessary to carry out the PCR using a thermal cycler by which exact temperature control is possible. The time required for setting the apparatus and reaction mixture at respective required temperatures increases with the number of cycles and, therefore, much time is required for the intended analysis.

For solving such problems, nucleic acid amplification methods which can be carried out under isothermal conditions have been developed. For example, the NASBA (nucleic acid sequence-based amplification) method (Non-patent document 4: J. Compton, et al., Nature, 350, 91-92 (1991)), the SDA (strand displacement amplification) method (Non-patent document 5: G. T. Walker et al., Proc. Natl. Acad. Sci. USA, 89, 392-396 (1992)), the 3SR (self-sustained sequence replication) method (Non-patent document 6: J. C. Guatelli, et al., Proc. Natl. Acid. Sci. USA, 87, 1874-1878 (1990)), the TMA (transcription-mediated amplification) method (Patent document 1: JP No. 3241717), the Qβ replicase amplification method (Patent document 2: JP No. 2710159), and the LAMP (loop-mediated isothermal amplification) method (Non-patent document 7: T. Natomi, et al., Nucleic Acids Research, 28, e63 (2000)), among others, are known as principal methods. According to these isothermal nucleic acid amplification methods, the primer extension, the primer annealing to a single-stranded chain extension product, and the subsequent primer extension are realized in a reaction mixture maintained at a constant temperature.

As for the methods using a DNA polymerase (SDA method and LAMP method) among these isothermal nucleic acid amplification methods, the SDA method uses dATPαS, in lieu of dATP, as a substrate for providing priming sites to serve as the starting points of amplification, and the LAMP method is designed so that the primer extension product may form a self-looped structure. Therefore, the SDA method encounters such problems as a low yield of substrate incorporation by enzyme and the resulting decrease in amplification efficiency, while the LAMP method has problems such that primer designing is difficult and it is difficult to design optimal primers for various test items. An isothermal nucleic acid amplification method by which these problems can be solved is thus demanded.

On the other hand, a method of synthesizing oligonucleotides, about 15 bases in length, utilizing, as priming sites, nicks formed on one strand of a double-stranded DNA using a nicking enzyme has also been reported (Non-patent document 8: Jeffery Van Ness, et al., Proc. Natl. Acad. Sci. USA, vol. 100, No. 8, 4504-4509 (2003)). However, this method cannot amplify nucleic acids exceeding 20 bases in length.

### SUMMARY OF THE INVENTION

Accordingly, it is a primary object of the present invention to provide a method which can solve the problems with the prior art isothermal amplification methods and enables gene expression or gene expression level quantification with ease and at low cost.

The inventors invented an isothermal amplification method which uses a DNA polymerase having strand displacement activity and a nicking enzyme. The present invention uses primers for amplification having a nicking enzyme recognition sequence-containing anchored sequence on the 5′ terminus side of a sequence recognizing a target sequence. As a result of extension of primers annealed to a target gene, a double-stranded DNA having nicking enzyme recognition sequences at both ends is formed. A nicking enzyme is caused to act on the double-stranded DNA to form a nick resulting from phosphodiester bond cleavage on one strand of the double-stranded DNA. By using the nicks formed as priming sites, it becomes possible to amplify the target sequence. A nick is formed in each nicking enzyme recognition sequence and, therefore, one nick is formed at each end of the double-stranded DNA. When a DNA polymerase and a substrate are allowed to act on these nicks, a DNA strand is synthesized. Owing to the strand displacement activity, the DNA strand extension proceeds isothermally without any thermal denaturation step, and the double-stranded DNA having a nicking enzyme recognition sequence at each end is regenerated. The DNA strand stripped off by the strand displacement activity hybridizes with amplification primers or a complementary DNA thereof to provide novel starting points for DNA synthesis.

Thus, according to an embodiment of the invention, nicking enzyme recognition sequences are introduced into a double-stranded DNA and nicks are formed on the double-stranded DNA by a nicking enzyme, whereby a DNA strand is caused to extend from the nicks formed for double-stranded DNA synthesis. The steps of nick formation on the double-stranded DNA by the action of the nicking enzyme and DNA strand extension with the nicks formed serving as priming sites are repeated substantially under isothermal conditions, namely at a temperature of 30° C. to 75° C., enabling isothermal amplification of the target sequence.

According to an embodiment of the invention, a nicking enzyme recognition sequence is introduced into each strand constituting a double-stranded nucleic acid and DNA strand extension is allowed to proceed from each nick site and, therefore, the region between the two nicks is the region to be amplified. The length of a nicking enzyme recognition sequence is generally 5-7 bases and a nick is formed in a phosphodiester bond within the recognition sequence or about 4 bases on the 3′ side from the recognition sequence. Nicking enzyme recognition sequences are introduced using primers having a base sequence about 15-25 bases in length and specifically hybridizing with a target gene, further having an appropriate linker sequence (0-5 bases long) on the 5′ end side according to need, and further having a nicking enzyme recognition sequence (5-7 bases long) on the 5′ end side. Therefore, the site of each nick formed is about 10-30 bases from the 3′ end of each primer. Since a DNA having a nicking enzyme recognition site at each end thereof is formed using such primers as mentioned above, the region between the two nicks is at least 21 bases in length. Therefore, the length of the DNA region amplified according to the present invention is at least 21 bases in length.

The nucleic acid amplification method of the invention makes it possible to design primers with ease using the ordinary substrate dATP and can realize an inexpensive and simple isothermal nucleic acid amplification method.

### BRIEF DESCRIPTION OF THE DRAWINGS

FIG. 1 is a flow chart illustrating a first aspect of the invention;

FIG. 2 is a flow illustrating the process in which the nicking enzyme recognition sequence-containing double-stranded DNA is obtained in the flow chart illustrating the first aspect of the invention;

FIG. 3 is a flow chart illustrating a second aspect of the invention;

FIG. 4 is a flow chart illustrating a third aspect of the invention;

FIG. 5 is an electrophoretic profile of the amplification product obtained in accordance with the flow chart illustrating the first aspect of the invention; and

FIG. 6 is a graphic representation of the results of real-time monitoring of the amplification process according to the flow chart illustrating a third aspect of the invention.

### DETAILED DESCRIPTION OF THE PREFERRED EMBODIMENTS

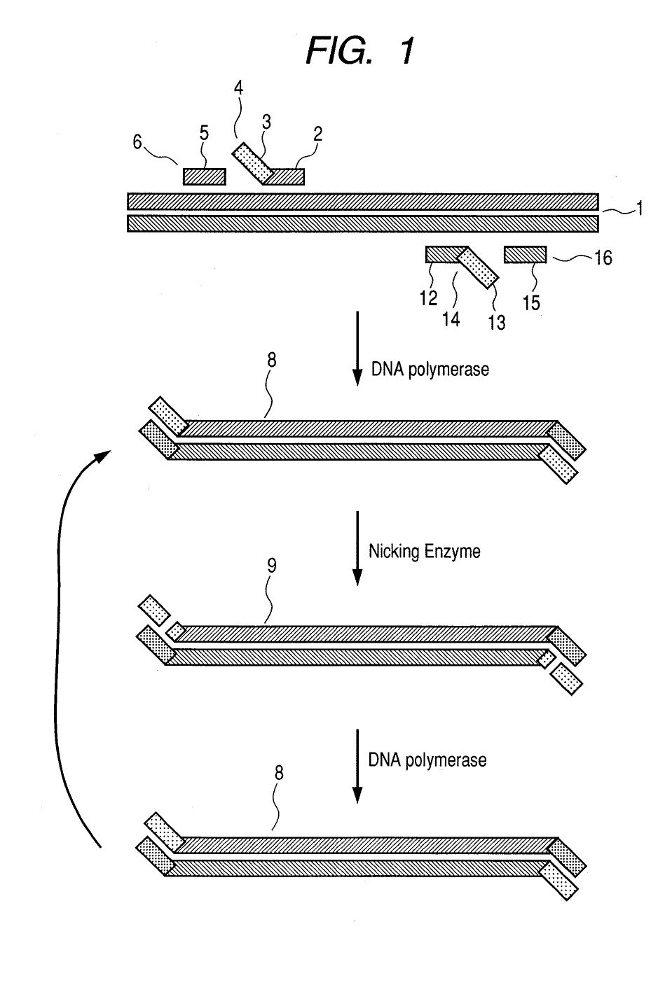

FIG. 1 is a flow chart illustrating a first aspect of the present invention. In this aspect, the invention relates to a method of amplifying and detecting a nucleic acid sequence, and the gene amplification method is characterized in that it comprises a first step of using a primer 4 having a sequence 2 having a sequence specific (complementary) to the base sequence of a target gene 1 and a sequence 3 attached to the 5′ end of the sequence 2 and noncomplementary to the base sequence of the target gene 1 and containing a nicking enzyme recognition sequence 3, a primer 6 capable of hybridizing with the target gene 1 on the side upstream of the primer 4 (on the side reverse to the direction of extension of the primer 4) and having a sequence 5 specific (complementary) to a base sequence of the target gene 1, a primer 14 containing a sequence 12 specific (complementary) to a base sequence of the target gene 1 and a sequence 13 attached to the 5′ end of the sequence 12 and noncomplementary to the base sequence of the target gene and containing a nicking enzyme recognition sequence, and a primer 16 capable of hybridizing with the target gene 1 on the side upstream from the primer 14 (on the side reverse to the direction of extension of the primer 14) and having a sequence 15 specific (complementary) to a base sequence of the target gene 1, together with the target gene 1 as a template, to cause extension of the primer 4, primer 14, primer 6, and primer 16, a second step of converting the nicking enzyme recognition sequence-containing double-stranded DNA 8 obtained as a result of the first step to a nick-containing double-stranded DNA 9 under the action of a nicking enzyme, and a third step of regenerating the nicking enzyme recognition sequence-containing double-stranded DNA 8 by causing a DNA polymerase having strand displacement activity to act, for DNA strand extension, on the nick-containing double-stranded DNA 9 the nicks of which serve as priming sites.

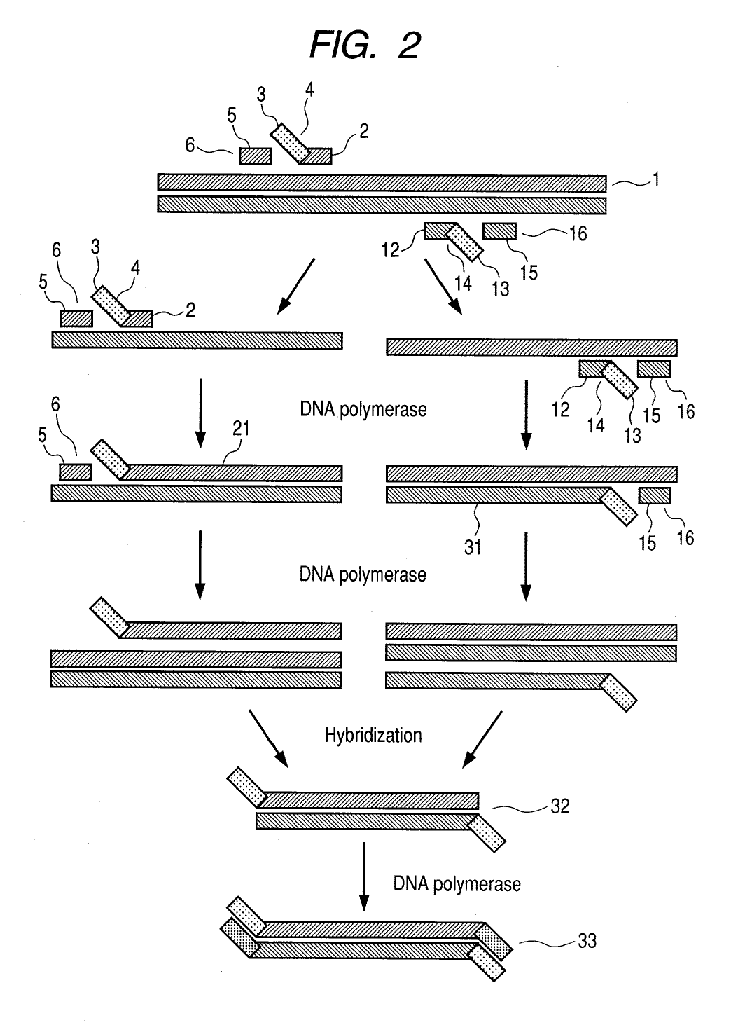

FIG. 2 shows in detail how the nicking enzyme recognition sequence-containing double-stranded DNA 8 is formed in the first step shown in the flow chart illustrating the first aspect of the invention. The primer 4 having a sequence 2 having a sequence specific to a base sequence of the target gene 1 and a nicking enzyme recognition sequence-containing sequence 3, and the primer 14 containing a sequence 12 having a sequence specific to a base sequence of the target gene 1 and a nicking enzyme recognition sequence-containing sequence 13 are extended with the target gene as a template. The primer 6 capable of hybridizing with the target gene on the side upstream from the primer 4 (on the side reverse to the direction of extension of the primer 4) and having a sequence 5 specific to a base sequence of the target gene 1 is extended while stripping off the primer 4-derived extension product 21 obtained in the initial step, and the primer 16 capable of hybridizing with a base sequence of the target gene 1 on the side upstream from the primer 14 (on the side reverse to the direction of extension of the primer 14) and having a sequence 15 specific to a base sequence of the target gene 1 is extended while stripping off the primer 14-derived extension product 31 obtained in the initial step. The primer extension products 21 and 31 hybridize with each other to form a double-stranded DNA 32, and the extension of the double stranded DNA 32 results in the formation of a nicking enzyme recognition sequence-containing double-stranded DNA 33. The double-stranded DNA 33 corresponds to the double-stranded DNA 8 in FIG. 1; thus, it is possible to introduce a nicking enzyme recognition sequence in each strand of the target gene.

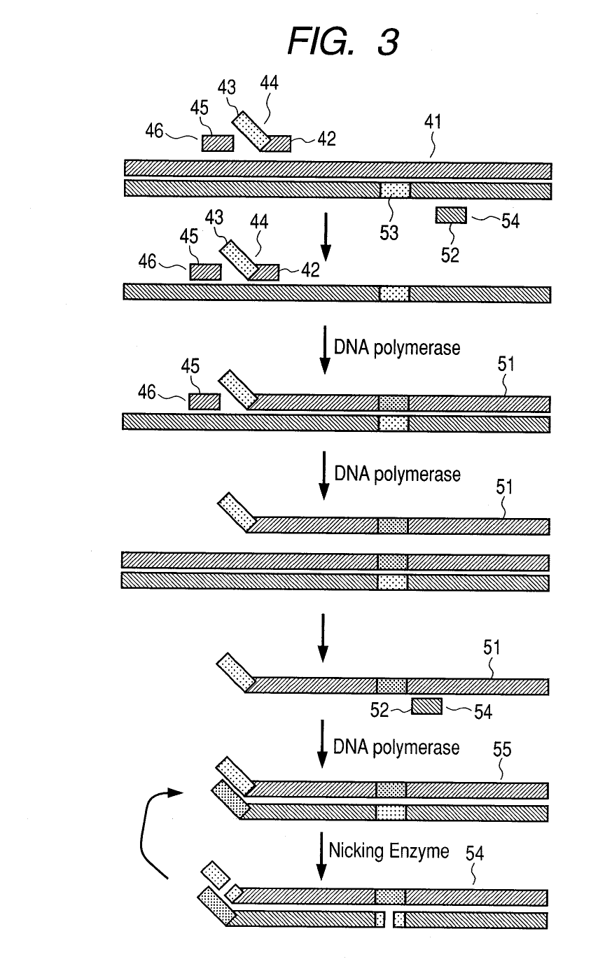

FIG. 3 is a flow chart illustrating a second aspect of the present invention. Using a primer 44 containing a sequence 42 having a sequence specific (complementary) to a nicking enzyme recognition sequence-containing target gene 41 and a nicking enzyme recognition sequence-containing sequence 43 attached to the 5′ end of the sequence 42 and noncomplementary to the base sequence of the target gene 41, a primer 46 capable of hybridizing with the target gene 41 on the side upstream from the primer 44 (on the side reverse to the direction of extension of the primer 44) and having a sequence 45 specific (complementary) to a base sequence of the target gene 41, and a primer 54 containing a sequence 52 specific (complementary) to the target gene 41, strand extension is carried out with the nicking enzyme recognition sequence-containing target gene 41 as a template. The primer 44 and primer 54 are designed so that they may hybridize with the target gene at respective sites apart on either side of the nicking enzyme recognition sequence 53 in the target gene 41.

The primer 46 having a sequence 45 specific (complementary) to the target gene 41 is extended while stripping off the primer 44-derived extension product 51. The primer 54 containing a sequence 52 having a sequence specific to the target gene 41 hybridizes with the stripped-off extension product 51, followed by strand extension to give a double-stranded DNA 55 having a plurality of nicking enzyme recognition sequences. Thereafter, a nicking enzyme is caused to act on the DNA 55, whereupon a nick-containing double-stranded DNA 54 is formed. The nicks formed serve as priming sites and when DNA strand extension is carried out under the action of a DNA polymerase having strand displacement activity, the nicking enzyme recognition sequence-containing double-stranded DNA 55 is regenerated. A nicking enzyme is again caused to act on the double-stranded DNA 53 to form the nick-containing DNA 54 and the DNA strand extension is carried out under the action of a DNA polymerase having strand displacement activity with the nicks serving as priming sites, whereupon the double-stranded DNA 55 is formed. By repeating these steps, it becomes possible to amplify a specific region of the target gene.

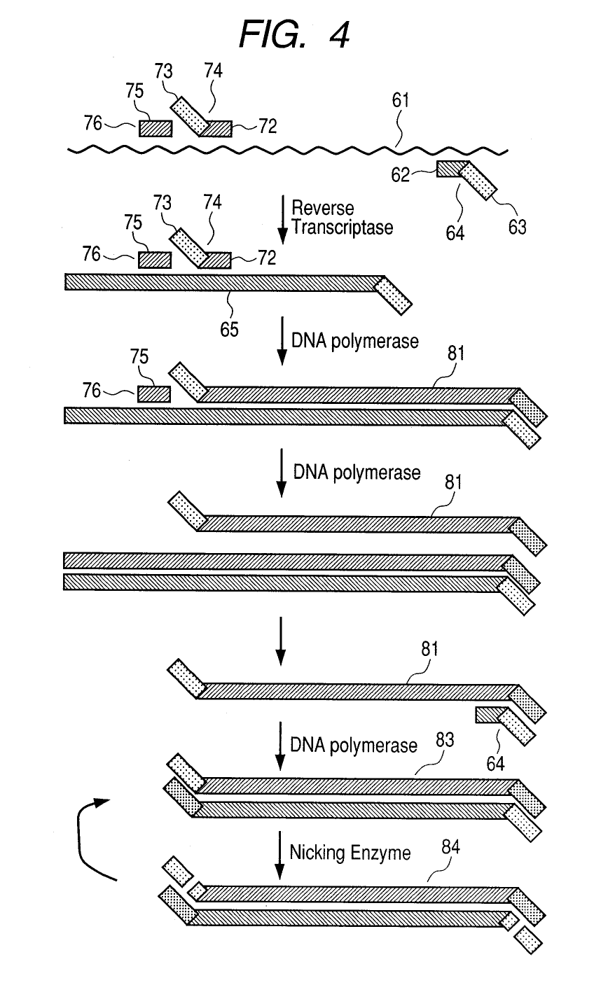

FIG. 4 is a flow chart illustrating a third aspect of the invention. A target RNA 61 to be amplified is subjected to reverse transcription reaction using a primer 64 having a sequence 62 having a sequence specific (complementary) to the target RNA 61 and further having a nicking enzyme recognition sequence-containing sequence 63 attached to the 5′ end of the sequence 62 and noncomplementary to the sequence of the target RNA 61. The reverse transcription reaction gives a nicking enzyme recognition sequence-containing cDNA 65. Using a primer 74 having a sequence 72 having a sequence specific (complementary) to the cDNA 65 (namely a sequence specific (complementary) to the complementary sequence relative to the target RNA) and having a nicking enzyme recognition sequence-containing sequence 73, and a primer 76 capable of hybridizing with the cDNA 65 on the side downstream from the primer 74 (on the side reverse to the direction of extension of the primer 74), the extension reaction is carried out with the cDNA 65 as a template. The primer 76 having a sequence 75 specific (complementary) to the cDNA 65 is extended while stripping off the primer 74-derived extension product 81. The primer 64 hybridizes with the extension product 81 stripped off and is extended to give a double-stranded DNA 83 having a plurality of nicking enzyme recognition sequences. Thereafter, a nicking enzyme is caused to act on the DNA 83 to give a nick-containing double-stranded DNA 84. The DNA strand extension is effected under the action of a DNA polymerase having strand displacement activity while the nicks formed serve as priming sites, whereupon the nicking enzyme recognition sequence-containing double-stranded DNA 83 is regenerated. A nicking enzyme is again caused to act on the double-stranded DNA 83 to form the nick-containing DNA 84 and the DNA strand extension is carried out under the action of a DNA polymerase having strand displacement activity with the nicks serving as priming sites, whereupon the double-stranded DNA 83 is formed. By repeating these steps, it becomes possible to amplify a specific region of the target RNA under substantially isothermal conditions, namely at a temperature of 30° C.-75° C.

As examples of the nicking enzyme which can be used in the practice of the invention, there may be mentioned BstNB (recognition sequence: GAGTC), N.AlwI (recognition sequence: GGATC), H. BbvCIA (recognition sequence: GCTGAGG), and N. BbvCIB (recognition sequence: CCTCAGC). As examples of the DNA polymerase having strand displacement activity which can be utilized in the practice of the invention, there may be mentioned Bst DNA polymerase, Vent DNA polymerase, Vent (exo-) DNA polymerase, Deep Vent DNA polymerase, Deep Vent (exo-) DNA polymerase, 9° Nm DNA polymerase, Terminator DNA polymerase, Klenow fragment of E. coli DNA polymerase I, Klenow fragment (3′→5′ exo-), and Phi29 DNA polymerase.

The following specific embodiments illustrate the present invention in more detail. These embodiments are, however, by no means limitative of the scope of the invention.

### First Embodiment

### 1. Primers Used in the First Embodiment

Forward side primer for nicking enzyme recognition sequence introduction:

(SEQ ID NO: 1)

5′-gtggt gagtc acaac ggtgg ctgga cccca gga-3′

Forward side primer:

| | |
|---|---|
| 5′-caagg gcctt tgcgt cag-3′ | (SEQ ID NO; 2) |

Reverse side primer for nicking enzyme recognition sequence introduction:

(SEQ ID NO: 3)

5′-gtggt gagtc acaac gcccc tgggc tcacc ccc-3′

Reverse side primer

| | |
|---|---|
| 5′-atctg ggaga caggc agg-3′ | (SEQ ID NO: 4) |

### 2. Composition of the Reaction Mixture Used in the First Embodiment

### Each Value in the Parentheses being a Final Concentration

Tris-HCl, pH 8.2 (15 mM), KCl (80 mM), (NH₄)₂SO₄ (5 mM), MgSO₄ (1 mM), MgCl₂ (5 mM), DTT (0.5 mM), dATP (0.3 mM), dCTP (0.3 mM), dGTP (0.3 mM), dTTP (0.3 mM), Triton X-100 (0.05%)

### 3. Enzyme Composition Used in the First Embodiment

Bst DNA Polymerase 8 U, N.BstNBI Nicking Enzyme 10 U

For confirming whether target gene amplification is possible according to the flow chart illustrating the first aspect of the invention, amplified product detection was carried out by electrophoresis. The human genome was used as a template, and the primers listed above under 1 were used for amplifying the insulin gene. The primer 4 used was a nicking enzyme recognition sequence introducing forward primer in which bases 1-10 from the 5′ end constituted a nicking enzyme recognition sequence-containing sequence, bases 11-15 constituted a linker sequence, and bases 16-33 constituted a nicking enzyme recognition sequence introducing forward primer comprising a sequence specific to the insulin gene. The primer 6 was a forward primer comprising a sequence specific to the insulin gene. The primer 14 was a nicking enzyme recognition sequence introducing reverse primer in which bases 1-10 from the 5′ end constituted a nicking enzyme recognition sequence-containing sequence, bases 11-15 constituted a linker sequence, and bases 16-33 constituted a sequence specific to the insulin gene. The primer 16 was a reverse primer comprising a sequence specific to the insulin gene.

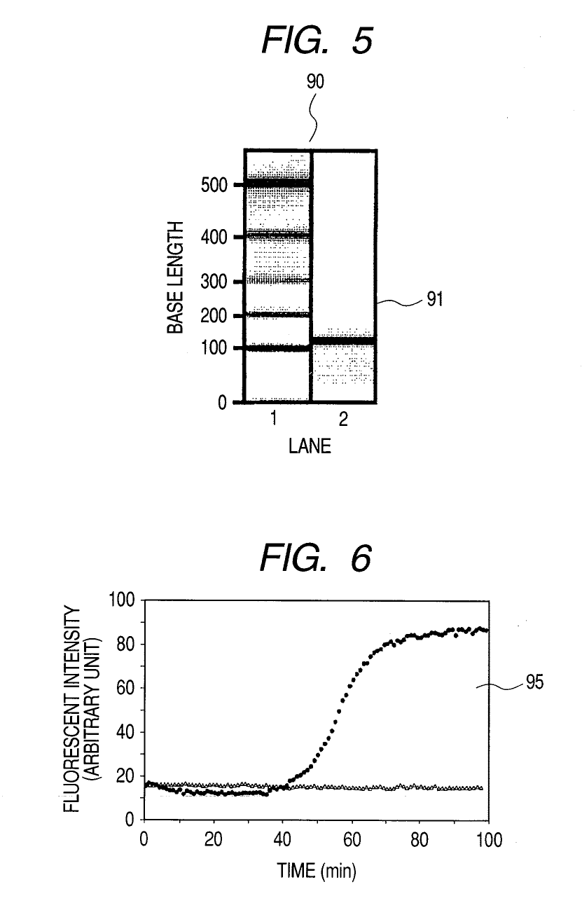

The composition of the amplification reaction mixture and the enzyme composition were as shown above under 1 and 2, respectively. The reaction mixture containing the human genome sample and primers was set on a heat block set at 60° C. and incubated for 120 minutes. Thereafter, the reaction product obtained was electrophoresed on a chip electrophoretic apparatus (SV1210, product of Hitachi High-Technologies). The results of the electrophoresis are shown as a gel image 90 in FIG. 5. Lane 1 indicates the results of electrophoresis of size markers, while lane 2 shows the result of electrophoresis of the reaction product. As a result, the expected 112 bpamplified product 91 could be detected. This result indicates that the desired amplification product was obtained by the present invention in its first aspect. Thus, it was confirmed that genes can be isothermally amplified in accordance with the present invention.

### Second Embodiment

### 1. Primers Used in the Second Embodiment

Nicking enzyme recognition sequence introducing primer for reverse transcription:

(SEQ ID NO: 5)

5′-catta gagtc tgttg tcgca agcac cctat cag-3′

Forward side primer for nicking enzyme recognition sequence introduction

(SEQ ID NO: 6)

5′-catta gagtc tgttg aatgc ctgga gattt ggg-3′

Forward side primer:

| | |
|---|---|
| 5′-tttct tggat aaacc cgc-3′ | (SEQ ID NO: 7) |

### 2.Molecular Beacon Probe used in the Second Embodiment Molecular Beacon Probe for Detection:

(SEQ ID NO: 8)

5′-cgacg tggga aatcg cgtgt agtat gggac gtcg-3′

### 3. Composition of the Reaction Mixture Used in the Second Embodiment

### Each Value in the Parentheses being a Final Concentration

Tris-HCl, pH 8.4 (35 mM), KCl (5 mM), NaCl (50 mM), (NH₄)₂SO₄ (5 mM), MgSO₄ (1 mM), MgCl₂ (5 mM), DTT (0.5 mM), dATP (0.3 mM), dCTP (0.3 mM), dGTP (0.3 mM), dTTP (0.3 mM), Triton X-100 (0.05%)

### 4. Enzyme Composition Used in the First Embodiment

Bst DNA Polymerase 16 U, N.BstNBI Nicking Enzyme 10 U

For checking whether a target gene can be amplified using the flow according to the third aspect of the invention, amplified product detection was carried out using a real-time detection system. The template used was RNA extracted from hepatitis C virus (HCV), and the primers specified above under 1 were used for reverse transcription of the viral RNA and for amplification. The primer 64 used was a nicking enzyme recognition sequence introducing primer for reverse transcription in which bases 1-10 from the 5′ end constituted a nicking enzyme recognition sequence-containing sequence, bases 11-15 constituted a linker sequence, and bases 16-33 constituted a nicking enzyme recognition sequence introducing primer for reverse transcription comprising a sequence specific to the viral RNA. The primer 74 was a forward primer for nicking enzyme recognition sequence introduction in which bases 1-10 from the 5′ end constituted a nicking enzyme recognition sequence-containing sequence, bases 11-15 constituted a linker sequence, and bases 16-33 constituted a sequence specific to the cDNA obtained from the viral RNA by the reverse transcription reaction. The primer 76 was a forward primer comprising a sequence specific to the cDNA obtained from the viral RNA by the reverse transcription reaction. The detection probe used was the molecular beacon probe described above under 2. The molecular beacon probe was labeled with FAM at the 5′ end and with BHQ1 at the 3′ end and, in the probe, bases 1-6 and bases 29-34 from the 5′ end constituted stem sequences, and bases 7-28 constituted a sequence for hybridization with the amplified product.

The composition of the reaction mixture and the specific examples of the enzyme composition were as shown above under 3 and 4, respectively. First, the reverse transcription reaction was carried out in the conventional manner using the revere transcription primers for cDNA production. Specifically, the reaction mixture containing the RNA sample to serve as a template and the reverse transcription primers was set on a heat block set at 42° C. and incubated for 60 minutes. Simultaneously, the reverse transcription reaction was carried out in the same manner as a negative control using a viral RNA-free sample. Thereafter, the reaction mixture containing the reverse transcription product, the reverse transcription primers and the two forward primers was set on a fluorescent microplate reader (Corona Electric) set at 63° C., and the time course of changes in reaction mixture fluorescent intensity was followed. For the negative control, the same measurement was carried out. The measurement results are shown in FIG. 6 in the form of a graph 95. The abscissa denotes the time, and the ordinate denotes the FAN fluorescent intensity. In the graph, the black dot plots show the amplification product-due fluorescent intensity data, and the white triangular plots show the viral RNA-free negative control-due fluorescent intensity data, and the respective results are shown in one and the same graph. The black dot plots show increases in FAN fluorescent intensity with time; thus, the amplification product formation in accordance with the flow chart illustrating the third aspect of the invention could be confirmed. On the other hand, the white triangular plots show no changes and thus it was found that no amplification product was formed. In view of the foregoing, the target RNA can be amplified and detected in accordance with the present invention.

The invention is useful in amplifying nucleic acids (DNAs or RNAs) in gene analysis. It can also be utilized as a method of judging the presence or absence of specific prokaryotic or eukaryotic genes and as a method of detecting nucleic acids amplified by that method.

What is claimed is:

1. A nucleic acid amplification method comprising the steps of:
using a first primer having a first sequence complementary to a part of one strand of an analysis target nucleic acid and a second sequence attached to the 5′ end of the first sequence, noncomplementary to said one strand and containing one nicking enzyme recognition sequence, a second primer comprising a third sequence complementary to a part of the region of said one strand on the 3′ end side from the region of the sequence thereof which is complementary to the first sequence, a third primer having a fourth sequence complementary to a part of the other strand of the target nucleic acid and a fifth sequence attached to the 5′ end of the fourth sequence, noncomplementary to the other strand of the target nucleic acid and having one nicking enzyme recognition sequence, a fourth primer comprising a sixth sequence complementary to a part of the region of the other strand on the 3′ end side of the sequence thereof which is complementary to the fourth sequence, and a DNA polymerase having strand displacement activity, to thereby cause the formation of a nucleic acid resulting from strand extension from the first primer and from the third primer, with the analysis target nucleic acid as a template;
causing nick formation in each extended strand of the resulting nucleic acid using a nicking enzyme; and
amplifying said nucleic acid utilizing the nick on each extended strand as a priming site.

2. The nucleic acid amplification method according to claim 1, wherein the first sequence, third sequence, fourth sequence, and sixth sequence each independently is 15-25 bases in length.

3. The nucleic acid amplification method according to claim 1, wherein the second sequence and fifth sequence each independently comprises an arbitrary linker sequence 1-5 bases in length.

4. A nucleic acid amplification method comprising the steps of:
using an analysis target nucleic acid containing one nicking enzyme recognition sequence, a first primer having a first sequence complementary to a part of one strand of the analysis target nucleic acid and a second sequence attached to the 5′ end of the first sequence, noncomplementary to said one strand and containing one nicking enzyme recognition sequence, a second primer comprising a third sequence complementary to a part of the region of said one strand on the 3′ end side of the sequence thereof which is complementary to the first sequence, a third primer having a fourth sequence complementary to a part of the other strand of the analysis target nucleic acid in a manner such that the fourth sequence and the first sequence may be positioned apart on either side of the nicking enzyme recognition sequence contained in the analysis target nucleic acid, and a DNA polymerase having strand displacement activity, to thereby cause the formation of a nucleic acid resulting from strand extension from the first primer and from the third primer, with the analysis target nucleic acid as a template;
causing nick formation in each extended strand of the resulting nucleic acid using a nicking enzyme; and
amplifying said nucleic acid utilizing the nick on each extended strand as a priming site.

5. The nucleic acid amplification method according to claim 4, wherein the first sequence, third sequence, and fourth sequence each independently is 15-25 bases in length.

6. The nucleic acid amplification method according to claim 4, wherein the second sequence contains an arbitrary linker sequence 1-5 bases in length.

7. The nucleic acid amplification method according to claim 1, wherein the length of the target nucleic acid region to be amplified is at least 21 bases in length.

8. The nucleic acid amplification method according to claim 1 in which the target nucleic acid is an RNA and which further comprises a step of introducing nicking enzyme recognition sequences into the target nucleic acid by the reverse transcription reaction.

9. The nucleic acid amplification method according to claim 1, wherein the amplification reaction is carried out under substantially isothermal conditions at a temperature of 30° C. to 75° C.


SEQUENCE LISTING (8 sequences)

SEQ ID NO: 1 - DNA, 33 bp, Artificial Sequence
  Description of Artificial Sequence Synthetic primer
gtggtgagtcacaacggtggctggaccccagga

SEQ ID NO: 2 - DNA, 18 bp, Artificial Sequence
  Description of Artificial Sequence Synthetic primer
caagggcctttgcgtcag

SEQ ID NO: 3 - DNA, 33 bp, Artificial Sequence
  Description of Artificial Sequence Synthetic primer
gtggtgagtcacaacgcccctgggctcaccccc

SEQ ID NO: 4 - DNA, 18 bp, Artificial Sequence
  Description of Artificial Sequence Synthetic primer
atctgggagacaggcagg

SEQ ID NO: 5 - DNA, 33 bp, Artificial Sequence
  Description of Artificial Sequence Synthetic primer
cattagagtctgttgtcgcaagcaccctatcag

SEQ ID NO: 6 - DNA, 33 bp, Artificial Sequence
  Description of Artificial Sequence Synthetic primer
cattagagtctgttgaatgcctggagatttggg

SEQ ID NO: 7 - DNA, 18 bp, Artificial Sequence
  Description of Artificial Sequence Synthetic primer
tttcttggataaacccgc

SEQ ID NO: 8 - DNA, 34 bp, Artificial Sequence
  Description of Artificial Sequence Synthetic probe
cgacgtgggaaatcgcgtgtagtatgggacgtcg